(19) United States
(12) Patent Application Publication
(10) Pub. No.: US 2010/0228361 A1
(43) Pub. Date: Sep. 9, 2010

(54) FLEXIBLE PRONG ADAPTOR FOR STUMP SOCKET FOR PROSTHETICS

(76) Inventor: Vladimir Radzinsky, Beverly Hills, CA (US)

Correspondence Address: CHRISTIE, PARKER & HALE, LLP, PO BOX 7068, PASADENA, CA, 91109-7068, US

(21) Appl. No.: 12/785,317

(22) Filed: May 21, 2010

Related U.S. Application Data
(63) Continuation-in-part of application No. 11/517,091, filed on Sep. 7, 2006, now Pat. No. 7,722,679.

(51) Int. Cl. A61F 2/80

(52) U.S. Cl. 623/33

(57) ABSTRACT

A socket adaptor for use in creating a laminated stump socket for attaching a prosthetic limb to a patient having a stump. It includes a separate main body and a plurality of flexible prongs that are permanently affixed to the main body. The main body is formed of relatively rigid and inflexible metal. The prongs are made of flexible sheet metal that can be repeatedly bent without weakening or breaking. Each prong is bendable by hand from a first position wherein the prong is substantially flat and extends radially outward in a plane from a base of the main body to a second position wherein the prong are conformed to fit to a stump socket.

### CROSS-REFERENCE TO RELATED APPLICATION(S)

This application is a continuation-in-part of prior application Ser. No. 11/517,091, filed Sep. 7, 2006.

### BACKGROUND OF THE INVENTION

The invention relates to adaptors for use with stump sockets for attaching a prosthetic limbs. More particularly, the invention relates to a socket adaptor that has flexible and repeatedly bendable prongs that allows the socket adaptor to be quickly and easily conformed to a stump socket which is attached to a prosthesis, such as a prosthetic limb.

Three prong laminating adaptors are often used in the prosthetic industry in the creation of laminated stump sockets. The laminated stump socket is fit over the residual limb or stump of the patient, and is configured to allow the prosthetic device to be attached thereto. Accordingly, the laminated stump socket must both fit comfortably on the residual limb or stump, and have sufficient structural integrity to create a reliable connection to the prosthetic device. Moreover, compatible hardware must be present to create the requisite connection to the prosthetic device.

To provide for these goals, currently available standard three prong adaptors have a threaded opening that allow a prosthetic connector or “pyramid” to be threaded thereto; and has prongs that are pre-formed into a downward arc to approximately conform to the distal end of the stump socket. These three prong adaptors are made of cast heat-treated stainless steel or other high strength strong metal, and thus are rigid and brittle. Prosthetists, in an attempt to make the patient more comfortable by making the stump socket fit better, will endeavor to bend the prongs to make them better conform to the stump socket. Attempts to bend the prongs, however, either result in an immediate fracture, or often create fractures that weaken the integrity of the adaptor and result in later breakage and shearing with continued usage.

The inventor previously developed a new socket adaptor that is the subject of allowed patent application Ser. No. 11/517,091, which instead of providing a socket adaptor that has prongs that are cast together with the prong body, has prongs formed of a more flexible and repeatedly bendable metal, compared to the more rigid socket adaptor body. The inventor's new socket adaptor that is the subject of this patent application provides further improvements that will allow prosthetists to more easily and quickly make a high strength laminated stump socket. Accordingly, the inventor's socket adaptor offers many advantages over the prior art socket adaptors.

### SUMMARY OF THE INVENTION

It is an object of the invention to provide a socket adaptor that facilitates secure attachment to a stump socket for a prosthetic limb or device within less time and effort.

It is another object of the invention to provide a socket adaptor that is more resistant to becoming detached from the socket.

It is a further object of the invention to provide a socket adaptor in which the flexible prongs can be bent and manipulated into any desired position numerous times without breaking or become weakened. Accordingly, the flexible prongs are made of a high strength sheet metal, including but not limited to titanium, stainless steel, and aluminum alloy, which possess sufficient strength and flexibility to fulfill the intended function.

It is yet another object of the invention to provide a socket adaptor which can be laminated to a socket for a prosthetic limb more quickly and with fewer steps.

An additional object of the invention is to provide a socket adaptor that contains an uninterrupted and continuous groove along the main body of the socket adaptor which allows for an evenly secured position of the overwrapped material prior to the lamination process

The invention is a socket adaptor, for use in creating a laminated stump socket for use with a patient having a stump, in attaching a prosthetic limb to the patient. The socket adaptor has a main body and a plurality of prongs. The prongs are made of sheet metal so that is repeatedly flexible and bendable to closely accommodate the stump socket. The socket adaptor is subsequently encapsulated with the laminated stump socket by overwrapping material. The main body allows a prosthetic connector to be attached thereto to allow the laminated stump socket to secure directly to the prosthetic limb.

To the accomplishment of the above and related objects the invention may be embodied in the form illustrated in the accompanying drawings. Attention is called to the fact, however, that the drawings are illustrative only. Variations are contemplated as being part of the invention, limited only by the scope of the claims.

### BRIEF DESCRIPTION OF THE DRAWINGS

In the drawings, like elements are depicted by like reference numerals. The drawings are briefly described as follows.

FIG. 1 is a perspective view, illustrating a prior art socket adaptor that is formed of a single piece of material, with generally inflexible and rigid and difficult to bend prongs.

FIG. 2 is a top plan view of the prior art socket adaptor of FIG. 1.

FIG. 3 is a perspective view, illustrating the prior art socket adaptor of FIG. 1 sitting atop a stump socket, prior to having its prongs bent down to better conform to the contours of the stump socket.

FIG. 4 is a perspective view, illustrating the prior art socket adaptor of FIG. 3 with overwrapping material shown placed around the prongs of the socket adaptor and partially covering its split clamp and screw, prior to being coated with resin.

FIG. 5 is a perspective view showing the laminated stump socket and overwrapped prior art socket adaptor with a clamping screw access grooves machined in place.

FIG. 6 is a perspective view illustrating an upper surface of an exemplary embodiment of a first socket adaptor of the invention.

FIG. 7 is a perspective view, illustrating a lower surface of the socket adaptor of FIG. 6.

FIG. 8 is a perspective view, illustrating a lower surface of another exemplary embodiment of a socket adaptor of the invention wherein a ring is used to sandwich the prongs in place to the main body.

FIG. 9 is a perspective view, illustrating the socket adaptor of FIG. 6 in an original, unbent state, prior to being fitted unto a stump casting.

FIG. 10 is a perspective view, similar to FIG. 9, except wherein the socket adaptor prongs are being bent to conform to the stump casting.

FIG. 11 is a perspective view, illustrating a prosthetic connector used with the socket adaptor of FIG. 6, positioned prior to being screwed in place.

FIG. 12 is a perspective view, illustrating the stump casting and socket adaptor prongs of FIG. 6 being covered with a graphite weave, prior to being coated with resin.

FIG. 13 is a top perspective view showing a exemplary embodiment of a second socket adaptor of the invention.

FIG. 14 is a bottom plan view showing the exemplary socket adaptor of FIG. 13.

FIG. 15 is a bottom perspective view showing the exemplary embodiment of the socket adaptor of FIG. 13.

FIG. 16 is a side plan view showing the exemplary socket adaptor of FIG. 13.

FIG. 17 is a top front view showing the exemplary socket adaptor of FIG. 13 sitting atop a stump socket, before its prongs are bent down to closely confirm in to the contours of the stump socket.

FIG. 18 is a perspective view showing the exemplary socket adaptor of FIG. 17, but with its prongs bent down to closely conform to the shape and contours of the stump socket.

FIG. 19 is a perspective view showing the exemplary socket adaptor of FIG. 18 overwrapped with material to secure the socket adaptor to the stump socket, prior to being coated with resin.

### DETAILED DESCRIPTION

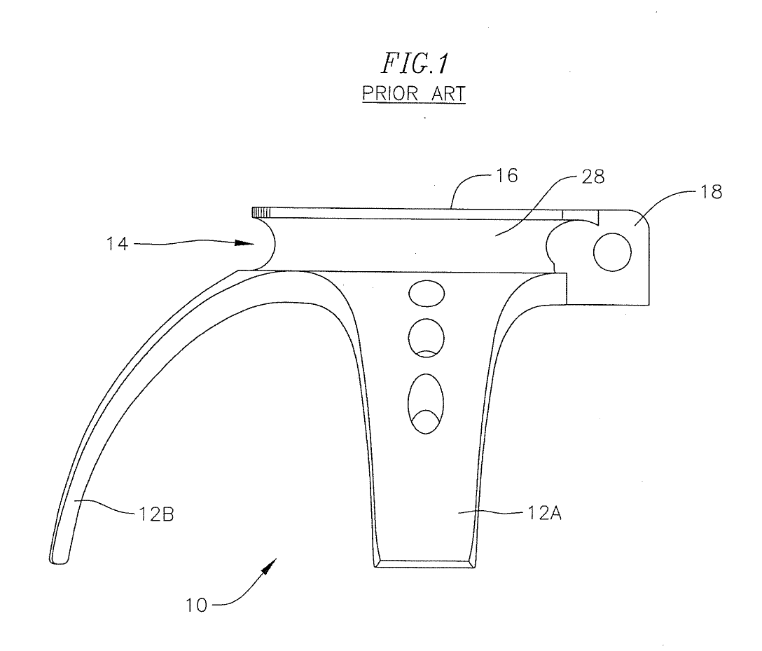

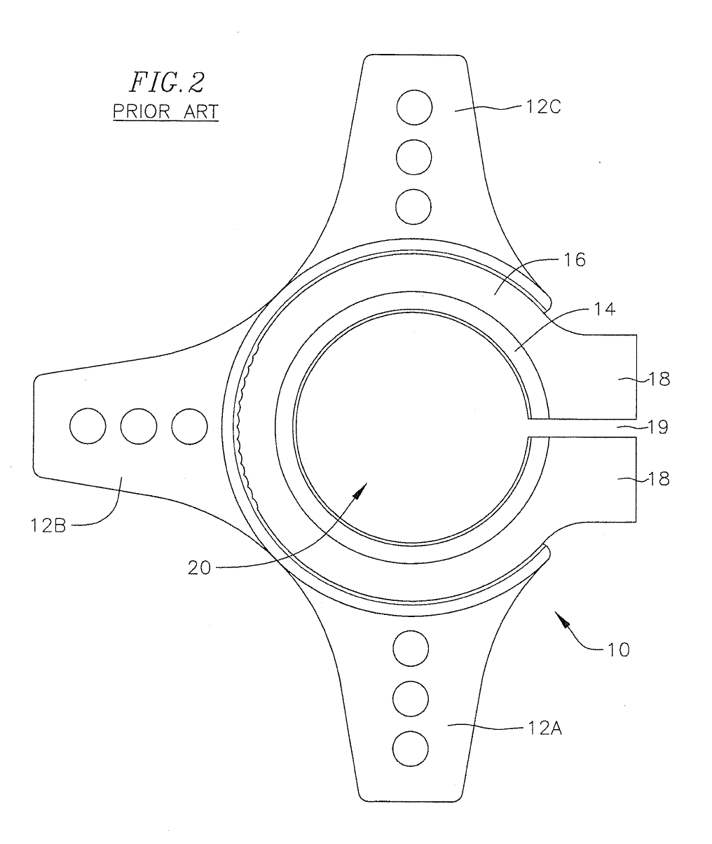

FIG. 1 is a diagrammatic perspective view, illustrating a prior art socket adaptor 10 that is formed of a single piece of material (e.g., by casting, stamping, machining, etc.), with generally inflexible and difficult to bend prongs 12A, 12B (12C shown in FIG. 2). The prongs 12A, 12B, 12C are formed together with a main body 14 with a top edge 16. In practice, the prior art socket adaptors are casts or machined as a single piece and the metallurgical properties, such as the tensile strength and level of rigidity of all portions, including the main body 14 and the prongs 12A, 12B, 12C, are the same. It has a split clamp 18 that is at generally the same level as the main body 14. A groove 19 passes through both halves of the split clamp 18. The main body 14 can have a groove 28 formed partially around its outer perimeter going around to join each half of the split clamp 18.

FIG. 2 is a top plan view of the prior art socket adaptor 10 of FIG. 1. As can be seen, the three prongs 12A, 12B, and 12C are generally positioned apart from each other by about 90 degrees, with the split clamp 18 positioned between prongs 12A and 12C. Thus, prongs 12A, 12B, and 12C, and split clamp 18 are positioned at about 6 o'clock, 9 o'clock, 12 o'clock, and 3 o'clock, respectively, around the main body 14. The main body 14 has a bore 22 formed therein.

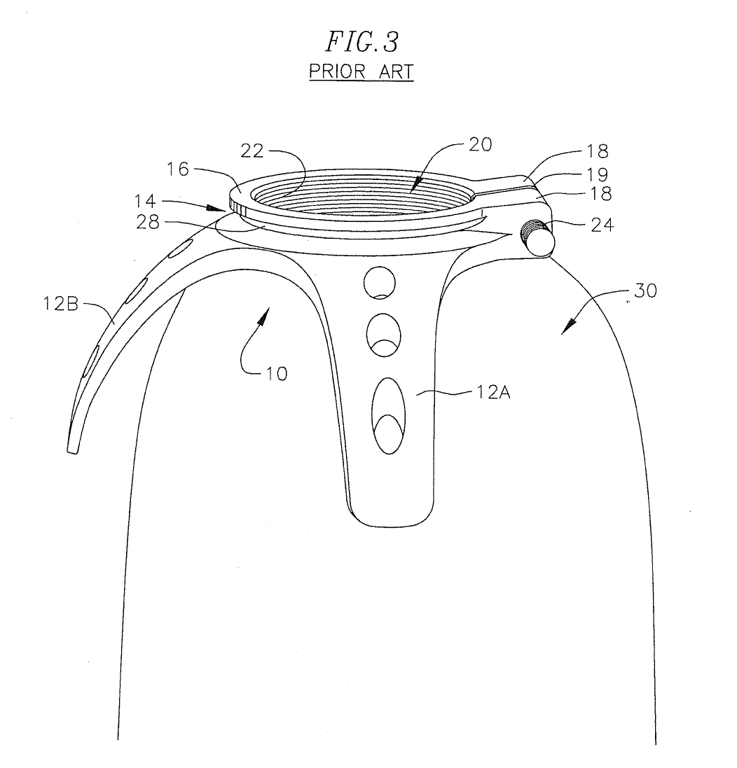

FIG. 3 is a diagrammatic perspective view, illustrating the prior art socket adaptor 10 of FIG. 1 sitting atop a stump socket 30, prior to having its prongs 12A, 12B (12C not shown) bent down to more closely conform to the contours of the stump socket 30. In practice, each stump socket 30 must be individually made to precisely fit the residual limb stump of a prosthetic user, and as a result, each stump socket will be unique in its internal and external size and shape. The bore 20 has threads 22 ion its inside and is adapted to engage with other accessories (not shown). A screw 24 is used to adjust the size of the gap 19 between the two portions of the split clamp 18 and the size of the threaded bore. After an accessory, e.g., a prosthetic connector, is screwed in place (not shown), the split clamp 18 is used to clamp down on accessory to securely retain it in place. As can be seen, due to the unique shape of each stump socket 30, the prongs 12A, 12B, 12C will not conform to the outer shape of stump socket 30, and must be bent to closely conform for a tight fit. Given the nature of the materials of which the socket adaptor is made of, e.g., stainless steel or titanium that is cast or machined from a block of material, the prongs have the same metallurgical properties as the main body and are very stiff and are difficult to bend and generally must be secured in a vice while a bending tool is used to bend the prongs. In order to establish a tight and conforming fit, the prosthetist must repeatedly adjust the prongs by bending in order to establish a close fit of the prior art socket adaptor 10 to the stump socket prongs. In the process of repeatedly bending the prongs, fractures can form, which compromises the structural integrity of the socket adaptor 10 and can lead to failure. Moreover, the split clamp 16 is more or less on the same level as the rim portion 14, which leads to problems as discussed below.

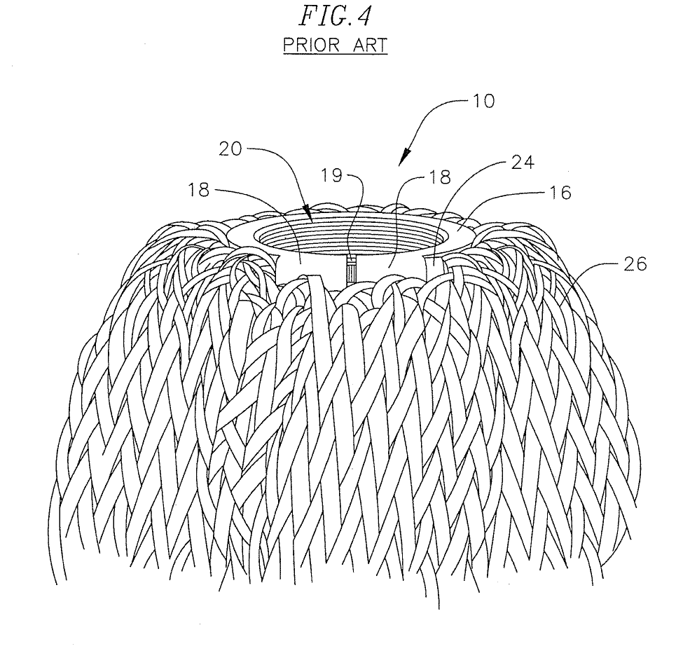

FIG. 4 is a diagrammatic perspective view, illustrating the prior art socket adaptor 10 of

FIG. 2 with overwrapping material 24 (prior to being coated with resin) shown placed around the prongs of the socket adaptor (not shown) and partially covering and crowding around the split clamp 18 and its clamping screw 24. As can be seen, the top edge 16 of the main body 14 barely extends above the level of the overwrapping material 26, which can for example comprise fiberglass, carbon fiber, Kevlar® (para-aramid synthetic fiber), and other fibers and materials.

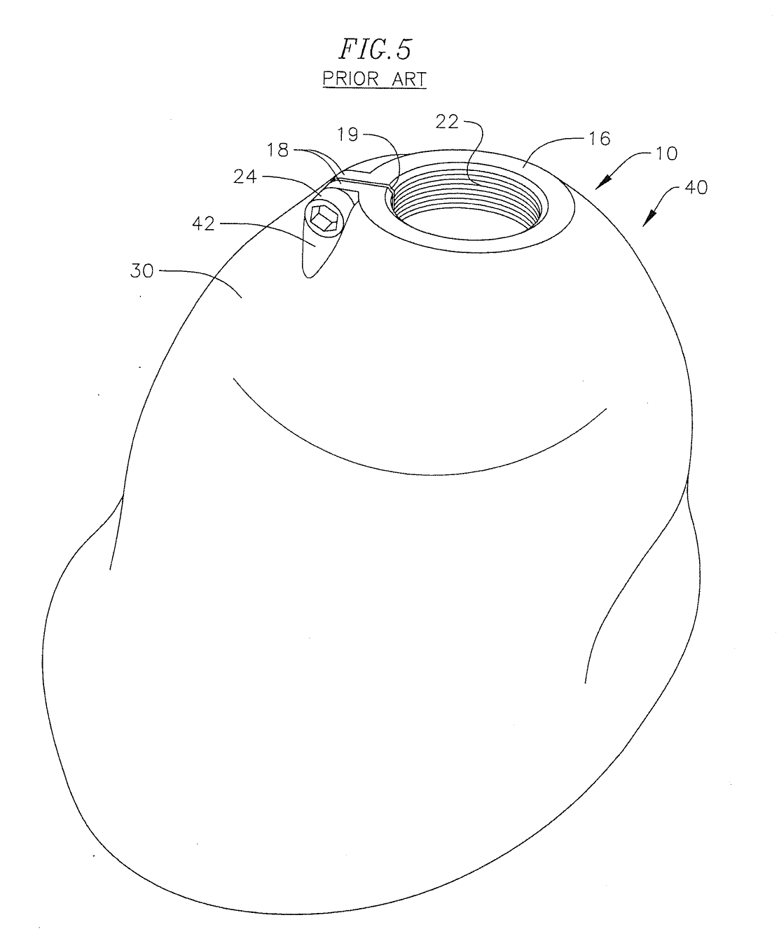

FIG. 5 is a diagrammatic perspective view showing a finished laminated stump socket 40 with its encapsulated prior art socket adaptor 10 covered by the resin cured overwrapping material 30. In order to gain access to the clamping screw 24, a clamping screw access groove 42 must be machined in the resin cured overwrapping material 30. Since resin often infiltrates the gap 19 and can also get on the threads of the screw 24, these parts must be cleaned of resin too. This requires additional time and labor, and the action of tightening the screw 24 can be further impeded by adhesion of the resin cured overwrapping material 30 to the socket adaptor 10. Positioning of the inflexible prongs of FIG. 1 causes disproportionate support of total surface area of residual limb increasing failure potential at the gap site between the split clamp 18.

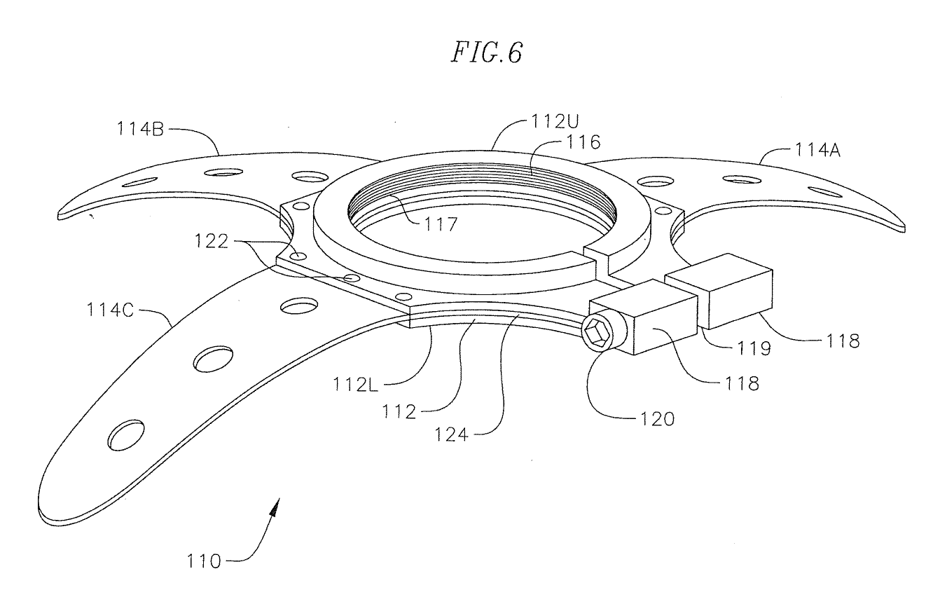

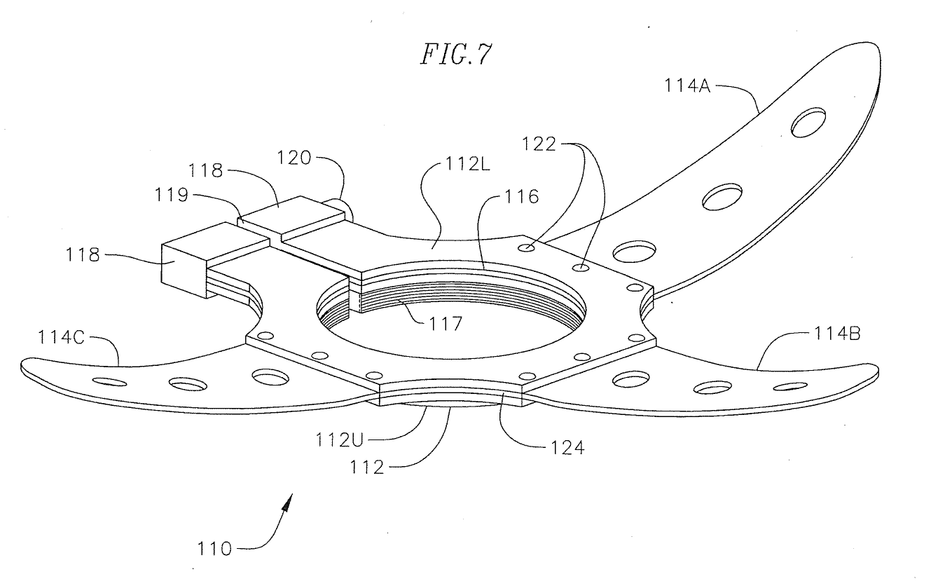

FIGS. 6 and 7 illustrate a first exemplary embodiment of a socket adaptor 110 for use in the creation of a laminated stump socket, for attaching a prosthetic limb or device to a patient. The socket adaptor 110 includes a main body 112, and three prongs 114A, 114B, and 114C extending outwardly from the main body 112. The main body 112 can be formed by casting or machining from a block of solid material, and is rigid and not flexible. The prongs 114 are each shown as being broad and flat, and substantially parabolic in shape, preferably having a curved extremity. However, other shaped and sized prongs can be used. The socket adaptor 110 may be manufactured such that the prongs 114A, 114B, and 114C initially extend in a co-planar configuration as the prongs 114A, 114B, and 114C are flexible and thus bendable-allowing them to be set as desired by the prosthetist in fitting the socket adaptor 130 to the stump socket of a patient. Due to the differences in the construction and metallurgical properties of the main body 112 compared to the prongs 114A, 114B, and 114C, the prongs 114A, 114B, and 114C can be freely bent without distorting the main body 112. To facilitate such bendability, the prongs are made of sheet metal, such as titanium, steel, stainless steel, aluminum alloys, or other high strength metals that are flexible and repeatedly bendable without substantially losing strength or because fractured or weakened. As used herein, the term “sheet metal” referral to relatively thin metal (less than 6 mm (0.25 inches) which by virtue of it nature (e.g., having been formed by repeated rolling), remains strong yet pliable and can be repeatedly bent, twisted, and deformed, such as to conform to a stump socket, without causing stress fractures or decreasing the structural integrity of the metal. For example, the use of sheet titanium material, and stainless steel provide extremely strong prongs which, unlike the prongs of the prior art socket adaptors, are able to be bent and re-bent repeated to the desired configuration to exactly fit to the contours of a stump socket without the need for bending tools and without causing damage to the prongs or distortion or damage to the main body. A suitable thickness for the sheet titanium has been discovered to be about 0.4 mm to about 1.2 mm, and more preferably about approximately 0.5 mm. Other thicknesses are also suitable and thus may also be used, such as about 0.6 mm to about 1.6 mm for stainless steel, and a thickness of about 0.8 mm to about 1.8 mm for aluminum alloy. The inventor has found that stainless steel, such as stainless steel 302 and stainless steel 304, and titanium 6-4 function well. In ASME (American Society of Mechanical Engineers) standards, a “strip” is 0.187″ (4.75 mm) and under in thickness and less than 24″ (609 mm) wide, while “sheet” is 0.187 (4.75 mm) and under in thickness and over 24″ (609 mm) wide. The inventor has found that stainless steel, such as stainless steel 302 and stainless steel 304, and titanium 6-4 function well. In ASME (American Society of Mechanical Engineers) standards, a “strip” is 0.187″ (4.75 mm) and under thick and less than 24″ (609 mm) wide, while “sheet” is 0.187″ (4.75 mm) and under thick and over 24″ (609 mm) wide. “Plate” is over 0.187″ (4.75 mm) thick and over 10″ wide (254 mm.) This is not the case with the socket adaptors of the prior art, including those of the type shown in FIGS. 1-5. The main body 112 has a lower surface 112L, and an upper surface 112U. A main bore 116 extends fully between the upper surface 112U and lower surface 112L. The main bore 116 is internally threaded 117, and may be selectively adjusted with an split clamp 118 that straddles a gap 119 that extends from the upper surface 112U and lower surface 112L and allows the main bore 116 to be slightly spread and narrowed. An adjustment screw 120 that regulates the magnitude of the split clamp 118. In particular, the adjustment screw 120 allows a device to be threaded into the main bore 116 and then prevented from unthreading by tightening the adjustment screw 120 to narrow the split clamp 118 and thus cause the main bore 116 to clamp upon the item. In construction, the three prongs 114A, 114B, and 114C can be secured to the main body 112 by use of rivets 122, by welding, adhesives, and/or clamping down the main body 112 on the prongs 114A, 114B, and 114C. In this regard, the main body 12 can be formed with a slot 124 into which ends of the prongs 114A, 114B, and 114C are inserted and then attached. As can be seen, the prongs 114A, 114B, and 114C and split clamp 118 are equally positioned around the main body, e.g., by 90 degrees, in a 3+1 orientation.

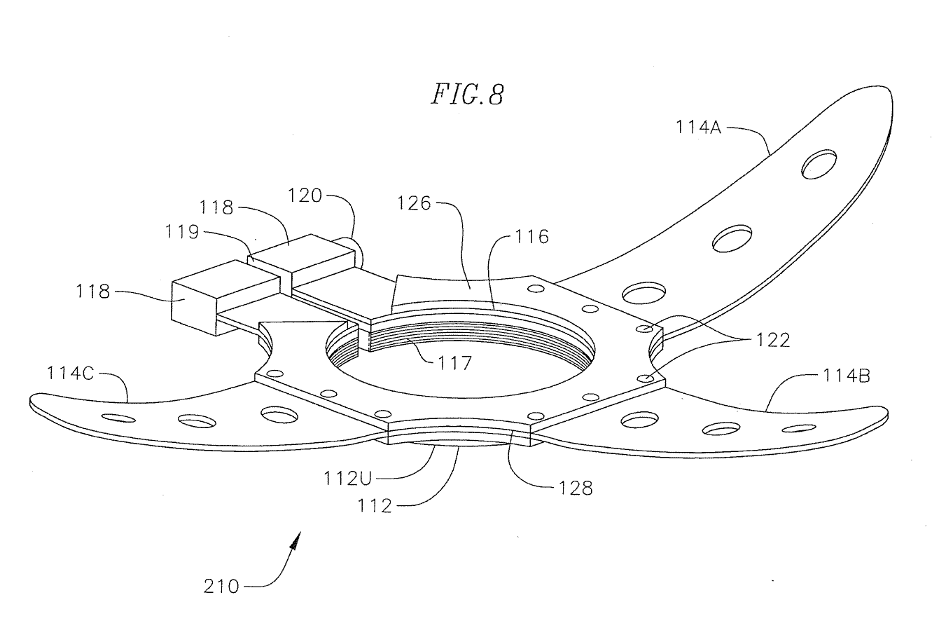

FIG. 8 shows an alternative embodiment of a socket adaptor 210, where in lieu of slot being formed in the main body 112 to receive ends of the prongs, a plate or ring 126 can be used to sandwich the prongs 114A, 114B, and 114C in place to the main body 112. Rivets 122 can be used to secure the prongs 114A, 114B, and 114C in place and if desired, adhesive can be additionally placed in a gap 128 between the plate or ring 126 and the main body 112. In other respects, this embodiment is similar to the embodiments of FIGS. 6 and 7.

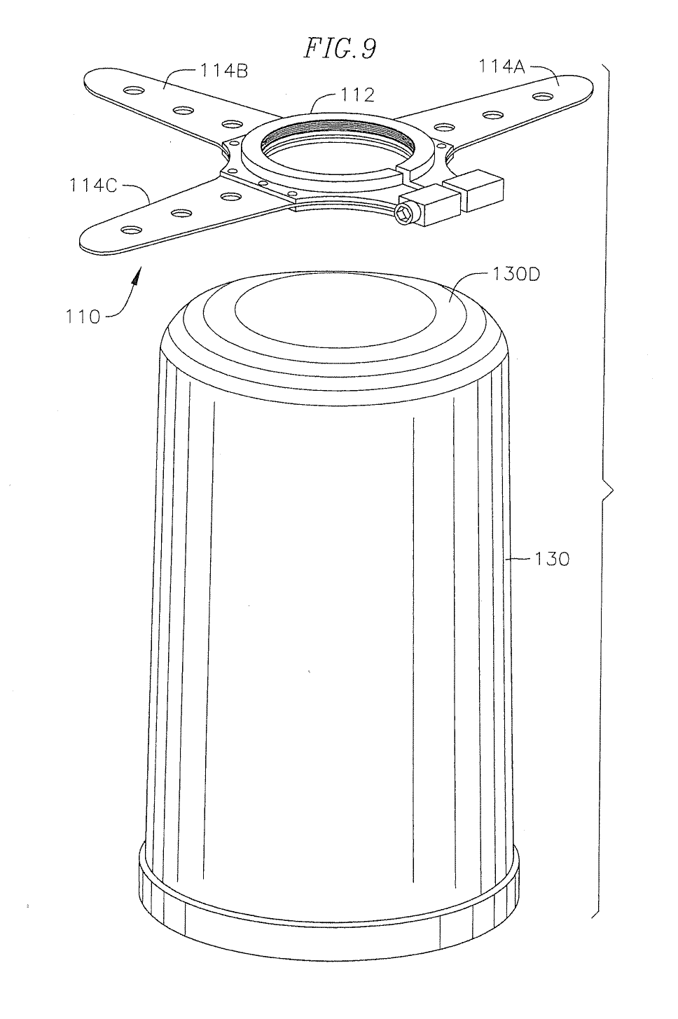

FIG. 9 illustrates the first exemplary socket adaptor 110 according to the present invention, wherein the prongs 114A, 114B, and 114C initially extend radially outwardly from the main body 112 in a common plane. Again, due to the differences in the construction of the main body 112 and the prongs 114, the prongs 114 can be freely bent without causing distortion to the main body 112. Socket adaptor 110 is shown positioned immediately above a stump casting a 130, having a distal end 130D. The stump casting 130 is created from a residual limb stump of a patient for which the laminated stump socket is intended. The creation of the stump casting 130 allows the prosthetist to work without requiring the patient to be present and thereby facilitates making a laminated stump socket that precisely fits the stump of the patient.

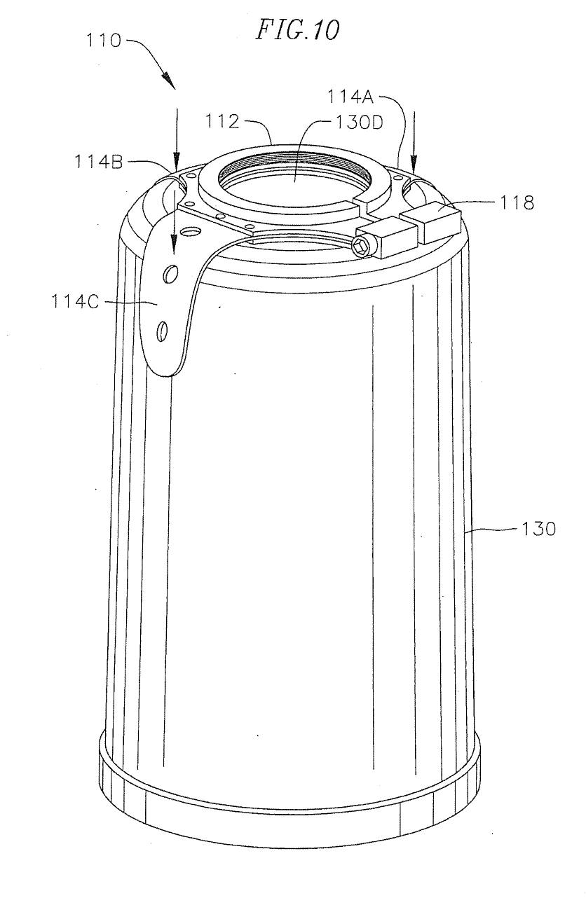

FIG. 10 illustrates the socket adaptor 110 being customized for the patient. In particular, the main body 112 is positioned against the distal end 130D of the stump casting 130, and the prongs 114 of the socket adaptor 110 previously illustrated in FIG. 9 are being bent downwardly to conform to the distal end 130D of the stump casting 130. Since the prongs 114A, 114B, and 114C of the present invention are made of sheet titanium, sheet stainless steel, or sheet metal of some other strong material, the bending can be repeated until a precise fit is obtained with close conformation of the prongs 114A, 114B, and 114C to the stump casting 130. As can be seen, one issue with the 3+1 format of the prongs 114A, 114B, and 114C and split clamp 118 of the socket adaptor 110 is that there is no prong to secure the socket adaptor 110 in the vicinity of the split clamp 118.

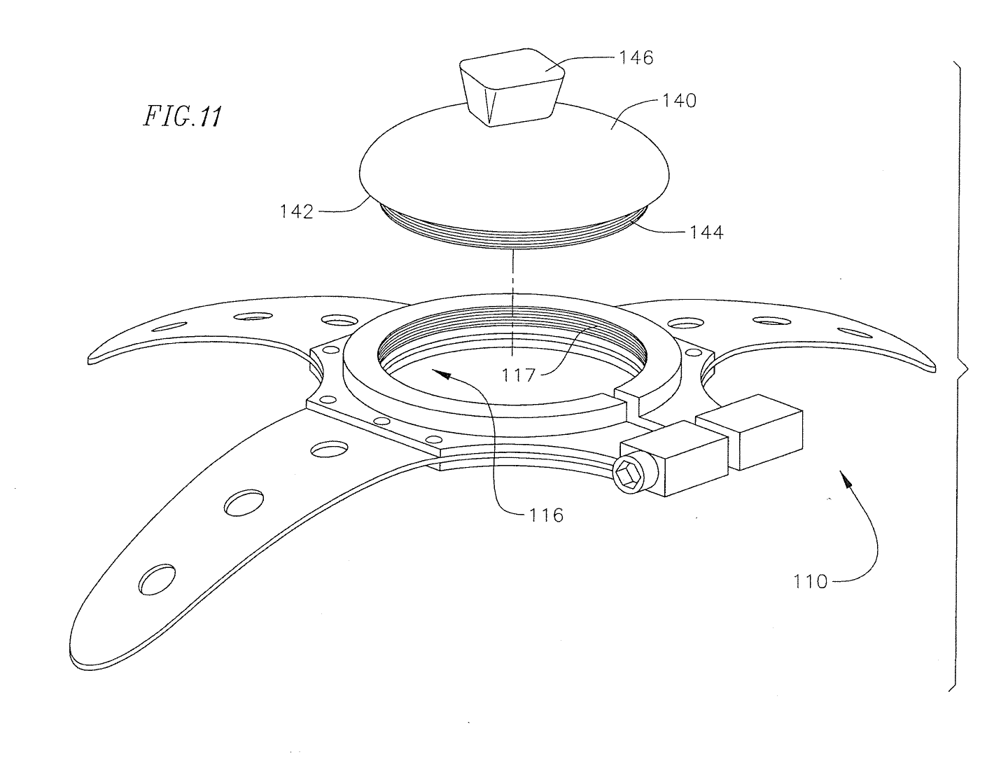

FIG. 11 illustrates a prosthetic connector, e.g., a pyramid plug 140 having a round base 142 with a threaded portion 144 and a pyramid plug 146 positioned above and ready to screw into the internally threaded 117 main bore 116 of the socket adaptor 110. The pyramid plug 146 allows connection of various prosthetic devices having hardware that is configured to attach thereto. The pyramid plug 146 may be substituted with other configurations that are adapted to connect to prosthetic devices having different connection hardware. It should be noted that according to a preferred embodiment, the main body 112 is made of solid titanium, solid stainless steel, or some other solid and generally inflexible material, as is the prosthetic connector 140. After the prosthetic connector 140 is screwed in place to the socket adaptor 110, the adjustment screw 120 can be tightening to narrow the adjustment opening 118 and thus cause the main bore 116 to clamp upon the threaded portion 144 of the prosthetic connector 140.

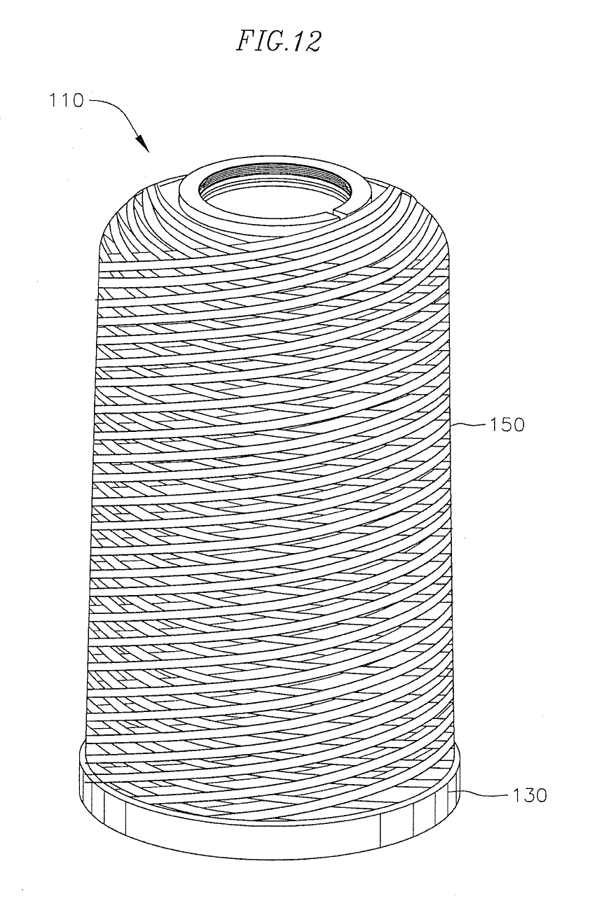

FIG. 12 illustrates the socket adaptor 110, fitted onto the stump casting 130, and covered with an overlaying material, such as graphite or carbon fiber mesh, fiberglass, Kevlar® (para-aramid synthetic fiber), and other fibers and materials 150. The overlaying material 50 is subsequently coated with resin to encapsulate the socket adaptor 110 and create a hardened, shell-like surface which is then removed from the stump casting 130 and is permanently formed to fit the stump of the patient. Most importantly, by using the socket adaptor 110, the laminated stump socket 166 thus created closely adapts to the residual limb stump of the patient for a comfortable fit, without sacrificing the structural integrity of the socket adaptor 110 encapsulated therein. Resin will be deposited under the prongs (not shown) on the stump casting 130 so that there is not direct contact of the metal prongs with the patient's stump. Alternatively, a layer of material can placed directly on at least areas of the stump casting 130, and the socket adaptor 110 can then be placed thereon, with overlaying material 150 subsequently applied and then soaked with resin to create the finished piece.

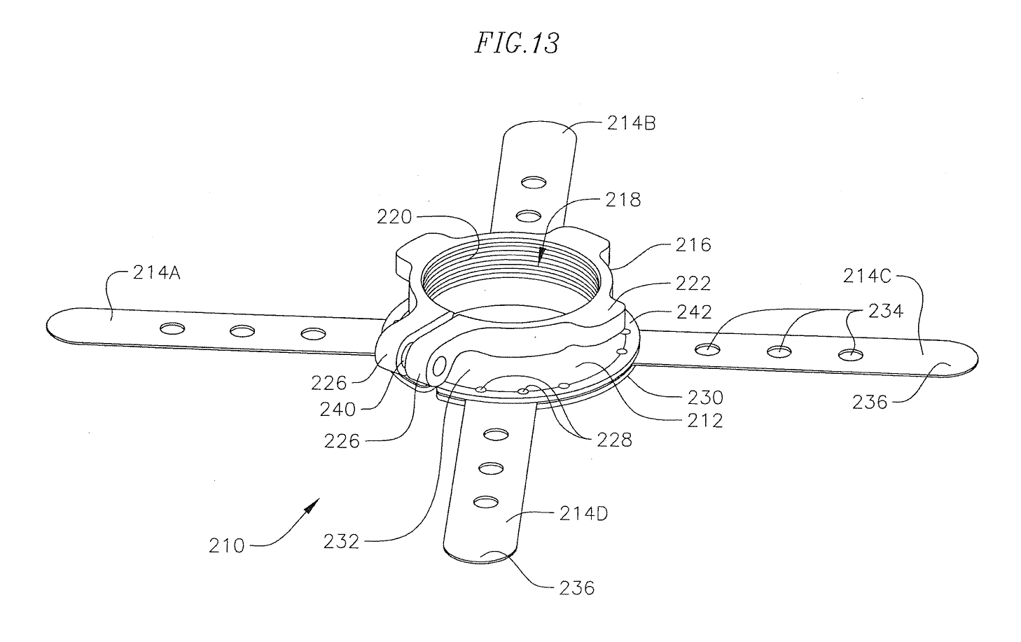

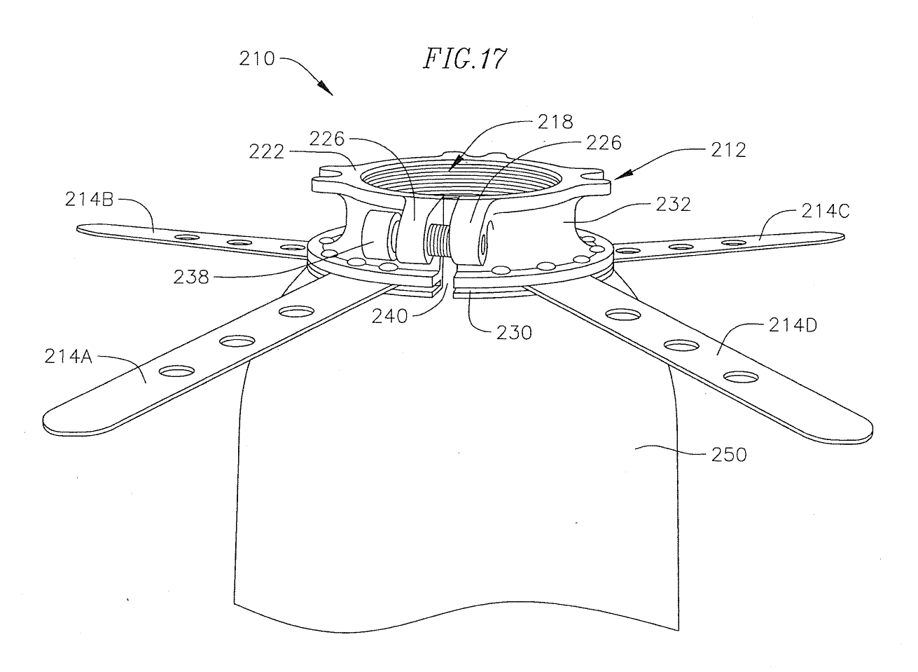

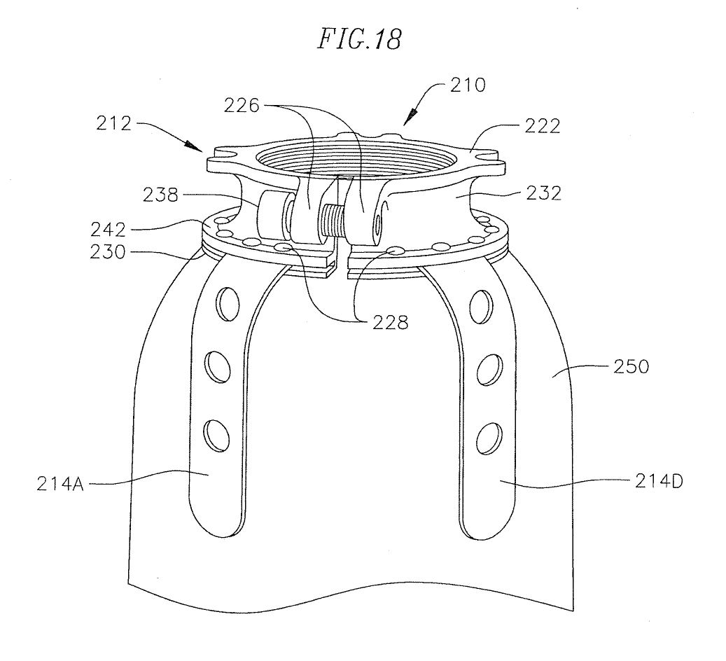

FIG. 13 is a diagrammatic top perspective view showing an exemplary embodiment of another socket adaptor 210 of the invention for use in the creation of a laminated stump socket, for attaching a prosthetic limb or device to a patient. The socket adaptor 210 includes a main body 112, and four prongs 214A, 214B, 214C, and 214D that extend outwardly from the main body 212. The main body 212 can be formed by casting or machining from a block of solid material, and is rigid and not flexible. The prongs 214A, 214B, 214C, and 214D are each flat, and substantially elongate in shape, preferably having rounded terminal ends 236. The socket adaptor 210 may be manufactured such that the prongs 214A, 214B, 214C, and 214D initially extend in a co-planar configuration because the prongs 214A, 214B, 214C, and 214D are flexible and thus bendable-allowing them to be set as desired by the prosthetist in fitting the socket adaptor 210 to the stump socket of a patient. Due to the differences in the construction and metallurgical properties (e.g., tensile strength, rigidity, etc.) of the main body 212 (being rigid) and the prongs 214A, 214B, 214C, and 214D (being flexible), the prongs 214A, 214B, 214C, and 214D can be freely bent without distorting the main body 212. To facilitate such bendability, the prongs are made of sheet metal, such as titanium, steel, stainless steel, aluminum alloys, or other high strength metals that are flexible and repeatedly bendable without substantially losing strength or because fractured or weakened. As used herein, the term “sheet metal” referral to relatively thin metal (less than 4.75 mm (0.187 inches)) which by virtue of it nature (e.g., having been formed by repeated been rolled), remains strong yet pliable and can be repeatedly bent, twisted, and deformed, such as to conform to a stump socket, without causing stress fractures or decreasing the structural integrity of the metal. For example, the use of sheet titanium material, and stainless steel provide extremely strong prongs which, unlike the prongs of the prior art socket adaptors, are able to be bent and re-bent repeated to the desired configuration to exactly fit to the contours of a stump socket without the need for bending tools and without causing damage to the prongs or distortion or damage to the main body. A suitable thickness for the sheet titanium has been discovered to be about 0.4 mm to about 1.2 mm, and more preferably about approximately 0.5 mm. Other thickness are also suitable and thus may also be used, such as about 0.6 mm to about 1.6 mm for stainless steel, and a thickness of about 0.8 mm to about 1.8 mm for aluminum alloy. The inventor has found that stainless steel, such as stainless steel 302 and stainless steel 304, and titanium 6-4 function well. In ASME standards, a “strip” is 0.187″ (4.75 mm) and under in thickness and less than 24″ (609 mm) wide, while “sheet” is 0.187 (4.75 mm) and under in thickness and over 24″ (609 mm) wide. “Plate” is over 0.187″ (4.75 mm) thick and over 10″ wide (254 mm.) This is not the case with the socket adaptors of the prior art, including those of the type shown in FIGS. 1-5. The main body 212 further has a sleeve portion 216 which defines a main bore 218 with internal threads 220. The main bore 218 extends fully between an upper surface 222 and lower surface 224 of the main body 212. The main bore 218 may be selectively adjusted with a split clamp 226 that straddles a gap 240 that extends between the upper surface 222 and lower surface 230 and allows the main bore 218 to be slightly spread and narrowed with an adjustment screw (shown in FIGS. 17 and 18) that regulates the magnitude of the split clamp 226. The split clamp 226 and its gap 240 are spaced up and away from the level of the prongs 214A, 214B, 214C, and 214D. The split clamp 226 and its gap 240 are positioned between two prongs so that expansion and contraction of the bore is unimpeded.

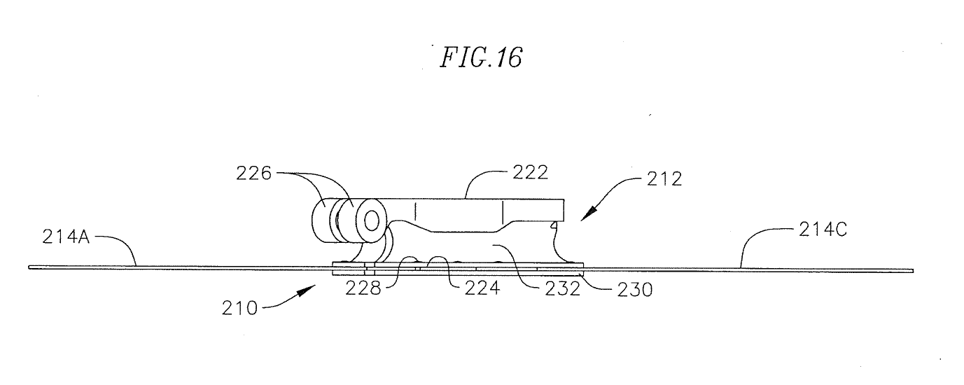

The adjustment screw allows a device, such as a pyramid plug 146 (such as shown in FIG. 11) to be threaded into the main bore 218 and then prevented from unthreading by tightening the adjustment screw to narrow the split clamp 226 and thus cause the main bore 218 to clamp upon the item. In construction, the four prongs 214A, 214B, 214C, and 214D can be secured to the main body 220 by use of rivets 226, by welding, adhesives, and/or clamping down the main body 212 on the prongs 214A, 214B, 214C, and 214D. In this regard, a plate or washer 230 can be provided to hat sandwich ends of the four prongs 214A, 214B, 214C, and 214D to an underside 224 of the main body 212, with rivets 228 securing the parts together. Adhesive, e.g., epoxy adhesive, can additionally be used to further secure the parts together and fill spaces between the underside 224 of the main body 212 and the washer 230 and the prongs 214A, 214B, 214C, and 214D. The prongs 214A, 214B, 214C and 214D and split clamp 226 are preferably equally positioned around the main body, e.g., by about 90 degrees and equally supports and distributes the loading forces, and the split clamp 226 is positioned above the level of the prongs 214A, 214B, 214C, and 214D, with an exposed throat area 232, as also shown in FIGS. 16-18. The throat areas 232 defines a continuous and uninterrupted groove along the main body which allows for an evenly secured position of the overwrapping material prior to the lamination process. A lower rim 242 of the main body 212 extends outwardly from the lower end of the throat 232, and it is through this lower rim 242 that the rivets 228 pass. Apertures 234 may be formed in the prongs 214A, 214B, 214C, and 214D to aid in bonding of the prongs to the stump socket. While the prongs are shown as being generally rectangular with rounded ends 236, they can be provided in different sizes and shapes as required. The threads 220 in the bore 218 will be located at a level above the level of the prongs and thus, are more free to expand and contract when the socket adaptor is secured to a stump socket.

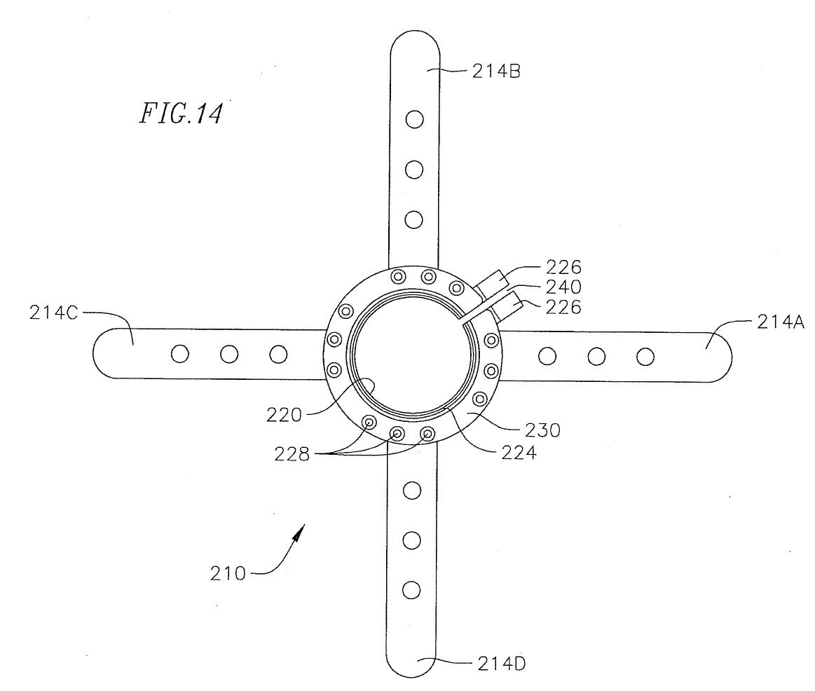

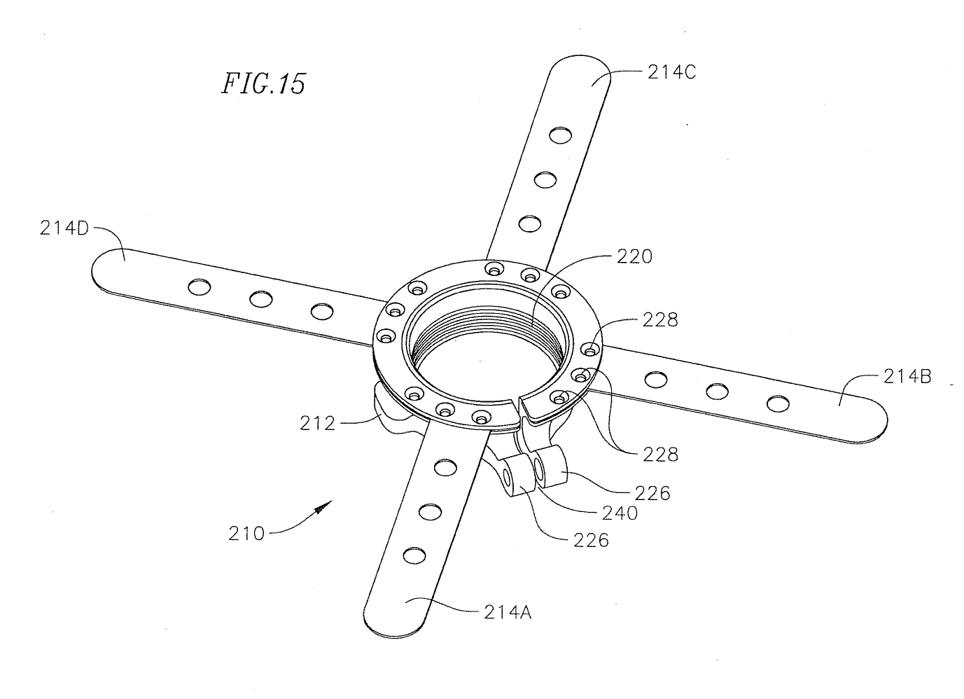

FIG. 14 is a bottom plan view and FIG. 15 is a bottom perspective view showing the exemplary socket adaptor 210 of FIG. 13, and best shows the plate or washer 230 that sandwiches ends of the four prongs 214A, 214B, 214C, and 214D to the underside 224 of the main body 212, with rivets 228 securing the parts together.

FIG. 16 is a side plan view showing the exemplary socket adaptor 210 of FIG. 13, and shows prongs 214A and 214C, the plate or washer 230 that sandwiches ends of the four prongs 214A, 214B, 214C, and 214D to the underside 224 of the main body 212, with rivets 228 securing the parts together. The throat 232 is shown in this view, which shows the split clamp 226 spaced well above the level of the prongs 214A, 214B, 214C, and 214D. Thus, as will be described below, when the socket adaptor 210 is formed together with overlaying material and resin to form the stump socket, the overlaying material will not cover over the split clamp 226.

FIG. 17 is a diagrammatic perspective view showing the exemplary socket adaptor 212 of FIG. 13 sitting atop a stump socket, before its prongs 214A, 214B, 214C, and 214D are bent down to closely confirm to the contours of the stump socket 250. The screw 238 is used to adjust the size of the gap 240 between the two ends of the split clamps 226, and thus adjust the diameter of the bore 218. As shown, the split clamps 226 sits above the level of the lower rim 242 and the prongs 214A, 214B, 214C, and 214D, with the throat 232 shown. The threaded bore 218 rises substantially above the level of the prongs.

FIG. 18 is a diagrammatic perspective view showing the exemplary socket adaptor 212 of FIG. 17, but with its prongs 214A, 214B, 214C, and 214D bent down to closely conform to the shape and contours of the stump socket 250. Due to the flexibility of the prongs 214A, 214B, 214C, and 214D a close fit can be established and the level of the split clamp 226 and screw 238 will be substantially raised up above the level of the lower rim 242, split clamp 226 and screw 238.

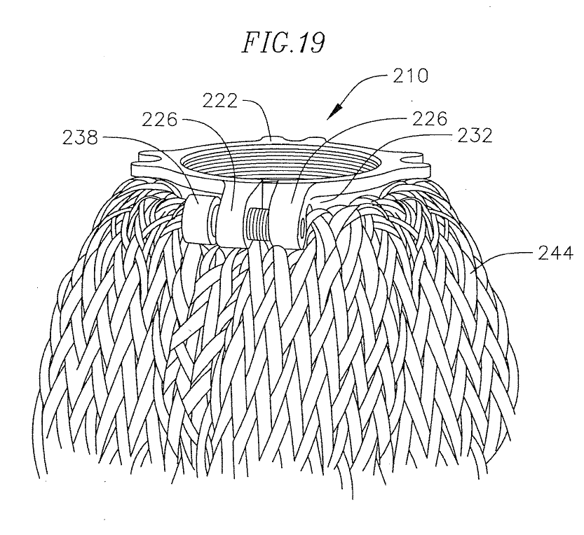

FIG. 19 is a diagrammatic perspective view showing the exemplary socket adaptor 210 of FIG. 18 after its prongs are covered with overwrapping material 244 to form a stump socket, but prior to being coated with resin. As can be seen, the overwrapping material 244 terminates around the throat 232, and its continuous and uninterrupted groove which allows for an evenly secured position of the overwrapping material prior to the lamination process, and compared to the prior art socket adaptor 10 shown used in FIG. 4 and the socket adaptor 210 shown used in FIG. 12, the level of the overwrapping material 244 will not reach up to the upper surface 222 of the socket adaptor 210 or cover the split clamp 226 or its screw 238. A result is that after being soaked with resin, the split clamp 226 and its screw 238 remain uncovered with overwrapping material and resin, and no additional labor is required to gain access to the screw 238 and split clamp 226.

Having thus described the exemplary embodiments of the present invention, it should be understood by those skilled in the art that the above disclosures are exemplary only and that various other alternatives, adaptations, and modifications may be made within the scope of the present invention. The presently disclosed embodiment is to be considered in all respects as illustrative and not restrictive. The scope of the invention being indicated by the appended claims rather than the foregoing description, and all changes which come within the meaning and range of equivalency of the claims are, therefore, intended to be embraced therein.

What is claimed is:

1. A socket adaptor for use in creating a laminated stump socket, comprising:
a separate main body comprising a first section and a second section being spaced apart to define a gap; and
a separate plurality of flexible prongs that are partly located inside the gap and permanently affixed to the main body, the prongs being made of flexible sheet metal so that they are substantially flat, flexible, and bendable and are adapted to be flexed and bent repeatedly to conform to be fitted to a stump socket without weakening the prongs, wherein the main body is made of metal having different material property than the sheet metal of the flexible prongs.

2. The socket adaptor as recited in claim 1, wherein the main body has a threaded main bore which is adapted to engage with a prosthetic connector which has a complimentary threaded base.

3. The socket adaptor as recited in claim 2, wherein the main body has an adjustment opening with a split clamp that allows the main bore to be slightly spread and narrowed, and an adjustment screw for the split clamp for selectively narrowing the main bore to clamp onto the threaded base of the prosthetic connector when threaded in the main bore.

4. The socket adaptor as recited in claim 3, wherein three flexible prongs are provided, and two prongs are spaced about 90 degrees apart from the adjustment opening on the main body, and the third prong is spaced about 180 degrees from the adjustment opening on the main body.

5. The socket adaptor as recited in claim 3, wherein four flexible prongs are provided, with the four prongs being spaced apart by about 90 degrees from each other, with the split clamp and adjustment screw being spaced above a level of the four flexible prongs, and wherein the four flexible prongs evenly distribute loading forces and help reduce possible failure within a gap in the split clamp.

6. The socket adaptor as recited in claim 6, wherein the main body is made of solid titanium.

7. The socket adaptor as recited in claim 6, wherein the flexible prongs are made of flexible sheet titanium and are affixed to the main body made of solid titanium, and wherein repeatedly bending, flexing and manipulation of the prongs does not fracture, crack or weaken the prongs.

8. The socket adaptor as recited in claim 7, wherein the titanium prongs have a thickness of about 0.4 mm to about 1.2 mm.

9. A socket adaptor for use in creating a laminated stump socket for attaching a prosthetic limb to a patient having a stump using a prosthetic connector, the socket adaptor comprising:
a main body comprising an upper surface, a lower surface, the main body being adapted for accepting a prosthetic connector for securing to the prosthetic limb, the main body being formed of relatively rigid and inflexible metal; and
a plurality of flexible prongs that are made of flexible sheet metal with a different material property than a material property of the main body that is more flexible and bendable than the main body such that the flexible prongs are able to be flexed and bent repeatedly to conform to be fitted to a stump socket without weakening the prongs, wherein the flexible prongs are permanently affixed to the lower surface of the main body, and wherein the prongs are adapted to be flexed to conform to be fitted to a stump socket.

10. The socket adaptor as recited in claim 9, further comprising a retention washer and rivets, which retention washer and rivets are used to permanently attach the prongs to the main body.

11. The socket adaptor as recited in claim 9, wherein the main body has a threaded main bore which is adapted to accept a complimentary threaded base of the prosthetic connector.

12. The socket adaptor as recited in claim 11, wherein the main body has an adjustment opening that allows the threaded main bore to be slightly spread and narrowed, and an adjustment screw for selectively narrowing the main bore to clamp onto the threaded base of the prosthetic connector when threaded in the threaded main bore.

13. The socket adaptor as recited in claim 12, wherein four flexible prongs are provided, with the four prongs being spaced apart by about 90 degrees from each other, with the adjustment opening on the main body being spaced above a level of the four flexible prongs.

14. The socket adaptor as recited in claim 9, wherein the flexible prongs are made of flexible sheet titanium and are affixed to the main body made of solid titanium, and wherein repeatedly bending, flexing and manipulation of the prongs does not fracture, crack or weaken the prongs.

15. The socket adaptor as recited in claim 14, wherein the prongs have a thickness of about 0.4 mm to about 1.2 mm.

16. A socket adaptor for use in creating a laminated stump socket for attaching a prosthetic limb to a patient having a stump using a prosthetic connector, the socket adaptor comprising:
a separate main body and a plurality of flexible prongs that are permanently affixed to the main body, the main body being formed of relatively rigid and inflexible metal and being adapted for accepting a prosthetic connector for securing to the prosthetic limb, the prongs being made of flexible sheet metal and being substantially flat, flexible and repeatedly bendable without weakening or breaking;
wherein each prong is bendable from a first position wherein the prong is substantially flat and extends radially outward from the main body in a plane defining the main body to a second position wherein a substantial portion of the prong is oriented transverse to the plane defining the main body and conform to be fitted to a stump socket, and wherein each prong is bendable by hand from the first position to the second position without using a tool; and
wherein the main body comprises a first section and a second section being spaced apart to define a gap, and wherein the plurality of flexible prongs are at least partly located inside the gap.

17. The socket adaptor as recited in claim 16, further comprising a retention washer and rivets, which retention washer and rivets are used to permanently attach the prongs to the main body.

18. The socket adaptor as recited in claim 16, wherein the main body has a threaded main bore which is adapted to engage with a prosthetic connector which has a complimentary threaded base.

19. The socket adaptor as recited in claim 18, wherein the main body has an adjustment opening that allows the main bore to be slightly spread and narrowed, and an adjustment screw for selectively narrowing the main bore to clamp onto the threaded base of the prosthetic connector when threaded in the main bore.

20. The socket adaptor as recited in claim 18, wherein four flexible prongs are provided, with the four prongs being spaced apart by about 90 degrees from each other, with the adjustment opening on the main body being spaced above a level of the four flexible prongs.

21. The socket adaptor as recited in claim 16, wherein the flexible prongs are made of flexible sheet titanium and are affixed to the main body made of solid titanium, and wherein repeatedly bending, flexing and manipulation of the prongs does not fracture, crack or weaken the prongs.

22. The socket adaptor as recited in claim 21, wherein the prongs have a thickness of about 0.5 mm to about 1.2 mm.
